SEQUENCE LISTING (6 sequences)

SEQ ID NO: 1 - DNA, 783 bp, Bacillus subtilis
  CDS (1)..(783)
atgggtatgaaaaagaaattgagtttaggagttgcttctgcagcactaggattagctttagttggaggaggaacatgggcagcatttaacgacattaaatcaaaggatgctacttttgcatcaggtacgcttgatttatctgctaaagagaattcagcgagtgtgaacttatcaaatctaaagccgggagataagttgacaaaggatttccaatttgaaaataacggatcacttgcgatcaaagaagttctaatggcgcttaattatggagattttaaagcaaacggcggcagcaatacatctccagaagatttcctcagccagtttgaagtgacattgttgacagttggaaaagagggcggcaatggctacccgaaaaacattattttagatgatgcgaaccttaaagacttgtatttgatgtctgctaaaaatgatgcagcggctgctgaaaaaatcaaaaaacaaattgaccctaaattcttaaatgcaagcggtaaagtcaatgtagcaacaattgatggtaaaaccgctcctgaatatgatggtgttccaaaaacaccaactgacttcgatcaggttcaaatggaaatccaattcaaggatgataaaacaaaagatgaaaaagggcttatggttcaaaataaatatcaaggcaactccattaagcttcaattctcattcgaagctacacagtggaacggcttgacaatcaaaaaggaccatactgataaagatggttacgtgaaagaaaatgaaaaagcgcatagcgaggataaaaat

SEQ ID NO: 2 - protein, 261 aa, Bacillus subtilis
MGMKKKLSLGVASAALGLALVGGGTWAAFNDIKSKDATFASGTLDLSAKENSASVNLSNLKPGDKLTKDFQFENNGSLAIKEVLMALNYGDFKANGGSNTSPEDFLSQFEVTLLTVGKEGGNGYPKNIILDDANLKDLYLMSAKNDAAAAEKIKKQIDPKFLNASGKVNVATIDGKTAPEYDGVPKTPTDFDQVQMEIQFKDDKTKDEKGLMVQNKYQGNSIKLQFSFEATQWNGLTIKKDHTDKDGYVKENEKAHSEDKN

SEQ ID NO: 3 - DNA, 759 bp, Bacillus subtilis
  CDS (1)..(759)
atgtttcgattgtttcacaatcagcaaaaggcgaagacgaaactgaaagttctgcttatctttcagctttcagtcattttcagtctgactgccgcaatatgcttacaattttccgatgatacaagcgctgcttttcatgatattgaaacatttgatgtctcacttcaaacgtgtaaagactttcagcatacagataaaaactgccattatgataaacgctgggatcaaagtgatttgcacatatcagatcaaacggatacgaaaggcactgtatgctcacctttcgccttatttgctgtgctcgaaaatacaggtgagaaacttaagaaatcaaagtggaagtgggagcttcataagcttgaaaatgcccgcaaaccgttaaaggatgggaacgtgatcgaaaaaggatttgtctccaatcaaatcggcgattcactttataaaattgagaccaagaaaaaaatgaaacccggcatttatgcatttaaagtatataaaccggcaggctacccggcaaacggcagtacatttgagtggtcggagcctatgaggcttgcaaaatgcgatgaaaaaccgacagtccctaaaaaagaaacaaagtcggacgtcaaaaaggagaatgaaacaacacaaaaagatataccggaaaaaacaatgaaagaagaaacatctcaagaagctgtaaccaaagaaaaagaaactcaatcagaccagaaggaaagcggggaagaggatgaaaaaagcaatgaagctgatcag

SEQ ID NO: 4 - protein, 253 aa, Bacillus subtilis
MFRLFHNQQKAKTKLKVLLIFQLSVIFSLTAAICLQFSDDTSAAFHDIETFDVSLQTCKDFQHTDKNCHYDKRWDQSDLHISDQTDTKGTVCSPFALFAVLENTGEKLKKSKWKWELHKLENARKPLKDGNVIEKGFVSNQIGDSLYKIETKKKMKPGIYAFKVYKPAGYPANGSTFEWSEPMRLAKCDEKPTVPKKETKSDVKKENETTQKDIPEKTMKEETSQEAVTKEKETQSDQKESGEEDEKSNEADQ

SEQ ID NO: 5 - DNA, 570 bp, Bacillus subtilis
  CDS (1)..(570)
atgaagctgatcagtaatattttatacgtgatcatctttactcttattattgtgctgacacttgtcgtgatttcaacacgttcatccgggggagagccggcagtgtttgggtatacgctgaaatcagttctgtcaggttcgatggagccggagttcaatacaggttccttaatattggtcaaagaaatcactgatgtgaaagagctccaaaaaggtgacgttattacatttatgcaggatgcaaatacggcggtcacccacagaattgttgacataacaaagcaaggagaccatttgttatttaaaacaaaaggtgataataatgcagcagctgattcagcgcctgtatcggacgaaaatgttcgcgcgcaatacacaggttttcagcttccatatgccggctatatgcttcattttgccagccagccgattggaacggctgtattattgattgttcccggcgtgatgctgttagtttacgcttttgtgacgatcagcagcgccattagagaaattgaaagaaagacaaaagccttggaaacagatacaaaggacagcaccatgtctact

SEQ ID NO: 6 - protein, 190 aa, Bacillus subtilis
MKLISNILYVIIFTLIIVLTLVVISTRSSGGEPAVFGYTLKSVLSGSMEPEFNTGSLILVKEITDVKELQKGDVITFMQDANTAVTHRIVDITKQGDHLLFKTKGDNNAAADSAPVSDENVRAQYTGFQLPYAGYMLHFASQPIGTAVLLIVPGVMLLVYAFVTISSAIREIERKTKALETDTKDSTMST

(19) United States
(12) Patent Application Publication
(10) Pub. No.: US 2002/0115184 A1
(43) Pub. Date: Aug. 22, 2002

(54) Antibiotic protein and method of production

(76) Inventors: Adam Driks, Chicago, IL (US); Axel Stover, Seattle, WA (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(21) Appl. No.: 09/757,749

(22) Filed: Jan. 10, 2001

Related U.S. Application Data
(63) Continuation of application No. PCT/US99/15488, filed on Jul. 9, 1999.
(60) Provisional application No. 60/092,397, filed on Jul. 10, 1998.

(51) Int. Cl. A61K 38/48; C07H 21/04; C12N 9/50; C12P 21/02; C12N 5/06; C12N 15/74

(52) U.S. Cl. 435/219; 424/94.63; 435/252.3; 435/320.1; 435/69.1; 536/23.2

(57) ABSTRACT

The invention provides an antibiotic protein having the properties of a native B. subtilis TasA protein. The invention also provides a method of translocating a desired protein across a membrane by expressing the desired protein as a fusion with a SipW recognition sequence in a membrane-bound genetic expression system. When a sporulating bacterium is employed as the membrane-bound expression system, the domain consisting of the desired protein is cleaved from the fusion protein and secreted into the resultant spores.

### CROSS REFERNCE TO RELATED APPLICATIONS

This is a continuation application of co-pending International Patent Application No. PCT/US99/15488, which was filed on Jul. 9, 1999, which designates the United States of America, and which claims priority to United States Provisional Patent Application No. 60/092,397, filed Jul. 10, 1998.

### STATEMENT AS TO RIGHTS TO INVENTIONS MADE UNDER FEDERALLY SPONSORED RESEARCH AND DEVELOPMENT

This invention was made with Government support under Grant Number GM53989-01 awarded by the National Institutes of Health. The United States Government may have certain rights in this invention.

### TECHNICAL FIELD OF THE INVENTION

The present invention relates to an antibiotic protein and a method of producing a protein.

### BACKGROUND OF THE INVENTION

The life cycle of all bacteria includes a vegetative phase in which the cells grow and divide. Normally, bacteria divide by building septa in the center of cells, forming roughly symmetrical daughter portions which then split to form independent cells. However, when starved, bacteria of the genera Bacillus and Clostridium can form spores. When such bacteria commit to forming a spore (called sporulation), they activate a special program of gene expression. This results in the synthesis of proteins required for constructing the spore. During sporulation, the cells construct a specialized asymmetrically positioned septum towards one end of the cell. This septum is composed of two inner membrane-derived membranes. Different sets of genes are expressed in the two asymmetrical cellular compartments. The smaller compartment (the forespore) ultimately becomes the spore, and the larger compartment (the mother cell) aids in spore formation. After the septum is made, the small compartment pinches off into a free protoplast surrounded by a double membrane. The spore is then encircled by a dark proteinatious coat. Finally, the mother cell lyses and releases the spore.

In contrast to vegetative cells, spores are tremendously resistant to environmental conditions. They can persist in extreme heat (including boiling conditions), and they can survive being frozen for prolonged periods of time. Additionally, they can survive without nutrients or even water. Spores can withstand acidic and basic conditions which would kill vegetative cells.

Considering their resiliency, spores can be formulated into a variety of compositions for commercial use, and spore-forming bacteria are of great use in disparate industries. For example, such bacteria have medical utility, chiefly as secretors of proteins for medical use (see, e.g., U.S. Pat. No. 5,728,571). Moreover, it is known that the spores of such bacteria display tropism for several types of cancerous tumors (Minton et al., FEMS Microbiol Rev., 17(3), 357-64 (1995); Fox et al., Gene Ther., 3(2), 173-78 (1996); Lemmon et al., Gene Ther. 4(8), 791-96 (1997)). In other applications, spores and spore-forming bacteria are used in agriculture or ecological control to treat soil, natural and industrial waste, and plants (see, e.g., U.S. Pat. Nos. 5,702,701, 5,464,766, 5,423,988, and 5,147,441). Strains of such bacteria are also useful agents for controlling insect populations, and spores are known to infect insects (see, e.g., U.S. Pat. Nos. 4,824,671, 5,202,240, 4,166,112, 4,206,281, and 5,556,784).

Several studies have reported on the antibacterial properties of strains of spore-forming bacteria and of antibiotic compounds produced or secreted from such bacteria (see, e.g., U.S. Pat. Nos. 5,147,441, and 5,753,222, and references cited therein). Despite this, few of the studies characterize such antibiotics, and it is difficult to produce them unless the bacteria are exposed to specific microenvironments. Thus, there is a need for improved methods for isolating and producing such antibiotics and for novel antibiotics produced from such bacteria.

In general, the utility of the spore-forming bacteria is due to the above-mentioned resiliency of their spores and their ability to secrete substances (e.g., enzymes, toxins, lipids, etc.) into their microenvironment. To this end, many novel strains of Bacillus and Clostridium have been isolated and engineered. Moreover, genetic systems have been developed to deliver exogenous genes to such bacteria for production and secretion of desired substances (see, e.g., U.S. Pat. Nos. 4,861,718, 5,429,950, 4,987,069, 4,952,508, and 5,624,849, and references cited therein). However, these methods and systems do not specifically concern the production of bacterial spores, as opposed to secretion into the microenvironment generally. Thus, there is a need for a method of incorporating a desired protein into a bacterial spore.

### BRIEF SUMMARY OF THE INVENTION

The invention provides an antibiotic protein having the properties of a native B. subtilis TasA protein. The invention also provides a method of translocating a desired protein across a membrane by expressing the desired protein as a fusion with a SipW recognition sequence in a membrane-bound genetic expression system. When a sporulating bacterium is employed as the membrane-bound expression system, the domain consisting of the desired protein is cleaved from the fusion protein and secreted into the resultant spores as well as out of the sporulating cells.

### DETAILED DESCRIPTION OF THE INVENTION

The present invention provides an antibiotic protein having the properties of a native B. subtilis TasA protein. At a concentration of at least about 2 μg/μl the protein will retard the growth of cultures of one or more of the following microbes: Microccocus luteus (4698), Staphylococcus epidermis (12228), Enterococcus faecalis (29212), E. coli (35218), E. coli (25922), Klebsiella pneumoniae (13883), E. coli (773465), E. coli (773813), E. coli (773671), Coagulase negative Staphylococcus sp. (X54017), Coagulase negative Staphylococcus sp. (174812), Coagulase negative Staphylococcus sp. (X54017), Klebsiella pneumoniae (773813), Klebsiella pneumoniae (773465), Enterobacter cloacae (538252-2), Enterobacter cloacae (618690), Agrobacterium tumifaciens (GV3101), Erwinia amylovora (EG321), Erwinia chrysanthamum (ACH150), Pseudomanas syringae pv. tomato (DC3000), Klebsiella pneumoniae (A95), Pseudomonas aurofaciens, Pseudomanas syringae pv. syringae (61), Erwinia (A1750), Pseudomonas putida, and Xanthomonas campestris (BP109). Preferably, the protein will retard the growth of substantially all of these microbes (e.g., at least about 80% of these microbes), or even all of these microbes. However, under the same conditions, the inventive protein will not significantly retard the growth of one or more of the following microbes: Staphylococcus aureus (33591), Enterococcus faecalis (52199), Streptococcus bovis (9809), Streptococcus pyogenes (19615), Pseudomonas aeruginosa (27853), Klebsiella pneumoniae (573266), Methicillin-resistant Staphylococcus aureus (S73185), Agrobacterium tumafaciens (GV3101 (harboring pB 1121)), and Pseudomanas aeruginosa (PAO). Additionally, the protein is an insecticide.

The inventive antibiotic protein can be an isolated or substantially purified Bacillus TasA protein, including a recombinant TasA protein. A cDNA encoding one mature full length TasA protein is set forth at SEQ ID NO:1, and an amino acid sequence of the protein encoded by this sequence is set forth at SEQ ID NO:2; however, the invention is not limited to these exemplary sequences. Indeed, genetic sequences can vary between different Bacillus species and strains, and this natural scope of allelic variation is included within the scope of the invention. Additionally and alternatively, the TasA protein can include one or more point mutations from the exemplary sequence or another naturally occurring TasA protein. Indeed, the TasA protein can also include other domains, such as epitope tags and His tags (i.e., the protein can be a fusion protein). Thus, within the context of the present invention, a TasA protein can, in some contexts, be or comprise an active fragment of these sequences or insertion, deletion, or substitution mutants. Preferably, any mutation is conservative in that it minimally disrupts the biochemical properties of the encoded TasA protein. Thus, where mutations are introduced to substitute amino acid residues, positively-charged residues (H, K, and R) preferably are substituted with positively-charged residues; negatively-charged residues (D and E) preferably are substituted with negatively-charged residues; neutral polar residues (C, G, N, Q, S, T, and Y) preferably are substituted with neutral polar residues; and neutral non-polar residues (A, F, I, L, M, P, V, and W) preferably are substituted with neutral non-polar residues.

The genes encoding such proteins typically are homologous to SEQ ID NO:1, e.g., they will hybridize to at least a fragment of SEQ ID NO:1 under at least mild stringency conditions, more preferably under moderate stringency conditions, and most preferably under high stringency conditions (employing the definitions of mild, moderate, and high stringency as set forth in Sambrook et al., Molecular Cloning: A Laboratory Manual, 2d edition, Cold Spring Harbor Press (1989)). Thus, a TasA gene is typically at least about 75% homologous to all or a portion of SEQ ID NO:1 and preferably is at least about 80% homologous to all or a portion of SEQ ID NO:1 (e.g., at least about 85% homologous to SEQ ID NO:1); more preferably the TasA gene is at least about 90% homologous to all or a portion of SEQ ID NO:1 (such as at least about 95% homologous to all or a portion of SEQ ID NO:1), and most preferably the TasA gene is at least about 97% homologous to all or a portion of SEQ ID NO:1. The TasA protein is similarly homologous to SEQ ID NO:2. Determining the degree of homology can be accomplished using any method known to those of skill in the art (e.g., Clusal or J. Hain method using PAM100 or PAM 250 residue weight table, etc.).

Where the protein is an isolated or substantially purified Bacillus TasA protein (or a derivative thereof), it can isolated from a species of Bacillus that produces the TasA protein, for example (but not limited to) B. subtilis 168 or any of the standard laboratory isolates of this strain or related species (e.g., B. subtilis natto). The protein can be isolated from spores or culture supernatant; however, a particularly good source of the TasA protein is from spores of a B. subtilis strain lacking the outer coat (e.g., B. subtilis gerE and/or cotE mutants). To isolate TasA from spores, they are first lysed and protein extracts obtained by standard methods. One form of mature TasA protein is identified as a 31 kDa protein having an N-terminal sequence AFNDIKSKD. Alternatively, an antibody recognizing TasA can be employed to separate TasA protein from such lysates by immunprecipitation or other immunopreparatory method.

Aside from isolating the native protein from Bacillus, TasA protein can be manufactured. For example, the protein can be synthesized using standard direct peptide synthesizing techniques (e.g., as summarized in Bodanszky, Principles of Peptide Synthesis (Springer-Verlag, Heidelberg: 1984)), such as via solid-phase synthesis (see, e.g., Merrifield, J. Am. Chem. Soc., 85, 2149-54 (1963); Barany et al., Int. J. Peptide Protein Res., 30, 705-739 (1987); and U.S. Pat. No. 5,424,398). As an alternative to solid phase protein synthesis, the inventive antibiotic protein can be manufactured by recombinant methods by transcribing an expression cassette encoding the TasA protein (i.e., any TasA protein described above) and translating the message. The expression cassette includes a nucleic acid encoding the TasA protein operably linked to a suitable promoter for driving the expression of the TasA-encoding sequence. Translation can occur in an organism or in vitro.

To facilitate the introduction of the cassette into a host organism, the invention provides a vector including the TasA cassette. The vector can be any type of genetic vector (e.g., virus, plasmid, cosmid, oligonucleotide, etc.) suitable for the host organism, many of which are known in the art and commercially available. One of skill in the art is able to employ standard techniques to introduce such vectors into the desired host organisms. Thus, for example, plasmids can be transferred by methods such as calcium phosphate precipitation, electroporation, liposome-mediated transfection, microinjection, viral capsid-mediated transfer, polybrene-mediated transfer, protoplast fusion, etc. Viral vectors can be transferred by infecting cells under conditions favorable to the virus. These and other methods are well-known in the art (see, e.g., Watson et al., Recombinant DNA, Chapter 12, 2d edition, Scientific American Books (1992)).

For producing TasA, the invention provides a genetic system harboring the TasA expression cassette. By expressing the TasA cassette, the system produces the antibiotic protein in biologically active form. The system includes the machinery necessary for transcribing and translating the protein, and it can be or comprise artificial elements (e.g., automated or in vitro systems) or living elements (e.g., eukaryotic or prokaryotic cells). While any organism or machine can be so employed to produce functional TasA, as TasA is a prokaryotic protein, typically the system will comprise a prokaryote (generally a Bacillus or E. coli strain). In one application, a cDNA encoding only mature TasA is introduced into such cells. The encoded protein does not include a secretory sequence, therefore the protein is isolated from such host organisms by lysing them and purifying protein fractions from crude lysate, if desired. Indeed, for some uses, crude lysate is sufficient. In other applications, a gene encoding the protein can include a leader sequence for promoting secretion of the protein from the cells. While many leader sequences are know, other leader sequences suitable for some applications can include about 10 or more residues from SEQ ID NO:2 or SEQ ID NO:4. Indeed, such leaders can include about 15 or more (e.g., about 20 or more) or even as may as about 25 or more (e.g., 40 or more) residues derived from these sequences (such as about 45 or more residues derived from the amino terminus of SEQ ID NO:4). More specifically, acceptable leaders can include residues 1-28 of SEQ ID NO:2 or residues 1-44 of SEQ ID NO:4. In such a situation, preferably the leader is cleaved during the processing of the protein to its mature form. Where the protein is secreted, it can be purified from the culture medium in which the host cells grow. Alternatively, where a sporulating host (e.g., Clostridium or Bacillus) is employed, the TasA protein can be isolated from spores, into which it will be incorporated if it is produced during sporulation.

Regardless of the manner by which the protein is produced, it can be incorporated into a composition, such as a pharmacological or agricultural composition, some of which are described herein. Thus, the protein can be used to treat a microbial culture. In accordance with such a method, a microbial culture is exposed to the protein (preferably within a pharmaceutical or other suitable composition) under conditions sufficient for the protein to (i.e., so as to) inhibit the growth of said culture.

The invention also provides a method of producing a desired protein such that it is translocated across a membrane. In accordance with the method, an expression cassette is introduced into a membrane-bound expression system that also includes the SipW protein. The expression cassette includes a nucleic acid encoding a fusion protein comprising a SipW recognition sequence domain and a domain consisting of the desired protein. A membrane separates the system into at least two compartments, a first of which contains all necessary transcription machinery and translation machinery, a SipW protein and the expression cassette. In accordance with the method, the expression cassette is transcribed within the system to produce a primary transcript, which is then translated into the fusion protein. In the presence of SipW, the fusion protein is processed to remove the SipW recognition sequence and to translocate the desired protein across the membrane and out of the first compartment.

To facilitate the use of the method, the present invention provides an expression cassette including a gene encoding a functional SipW protein operably linked to a promoter. A cDNA encoding one full length SipW protein is set forth at SEQ ID NO:5, and the corresponding amino acid sequence of this protein is set forth at SEQ ID NO:6; however, the invention is not limited to these exemplary sequences. Indeed, the SipW protein can reflect the natural scope of allelic variation and expected mutant variations highlighted above in connection with the discussion of the TasA gene and protein. Thus, a sipW gene is typically at least about 75% homologous to all or a portion of SEQ ID NO:5 and preferably is at least about 80% homologous to SEQ ID NO:5 (e.g., at least about 85% homologous to SEQ ID NO:5); more preferably the sipW gene is at least about 90% homologous to SEQ ID NO:5 (such as at least about 95% homologous to SEQ ID NO:5), and most preferably the sipWgene is at least about 97% homologous to SEQ ID NO:5.

Regardless of the sequence of the sipW gene employed, the presence of functional SipW can be assessed using TasA. An expression cassette encoding pro-TasA (i.e., containing the N-terminal amino acids cleaved from the mature protein) is introduced into the membrane-bound genetic system harboring the putative SipW cassette. Thereafter, the system is assayed for the presence of mature 31 kDa TasA protein. Because processing and secretion of TasA is dependent on functional SipW, the presence of the 31 kDa species indicates that the putative SipW gene encodes a functional protein. In contrast, the immature pro-TasA protein is 34 kDa. Hence the presence of a predominant amount of 34 kDa species indicates that the putative sipW gene does not encode a functional SipW protein.

In accordance with the inventive method, an expression cassette encoding the desired protein is introduced into the SipW-containing membrane-bound genetic system. The sequence encoding the desired protein also encodes a SipW recognition sequence domain (i.e., the cassette encodes a fusion protein). Desirably, the gene is constructed to attach the SipW recognition domain to the amino terminus of the desired protein, as the recognition sequences of SipW-processed proteins (e.g., YqxM and TasA) are so situated. The SipW recognition domain is any domain that results in SipW-dependent processing of the protein. Proper SipW-dependent processing has two characteristics, and the presence of both characteristics identifies any amino acid sequence as a SipW recognition domain. The first characteristic is that in the presence of SipW, the SipW recognition domain will be cleaved from the fusion protein during processing. Thus, the absence of the putative signal from the N-terminus of the processed protein indicates that it is a SipW recognition sequence. The second characteristic is that in the presence of SipW, the desired protein is translocated across the membrane. While those of skill in the art will be able to design suitable SipW recognition domains for use in the inventive method, it can be derived from SEQ ID NO:2 or SEQ ID NO:4 as discussed above.

Within the genetic system, in the presence of SipW, the SipW recognition domain is cleaved from the immature protein, and the mature protein is exported across the membrane. The membrane (and indeed, the entire genetic system) can be artificial. For example, an artificial membrane can define into two (or more) compartments. One component can contain the transcription and translation machinery. After the protein is produced, it is then translocated into the other compartment. In other embodiments, the system employs living cells, typically prokaryotes (e.g., E. coli, Clostridium, Bacillus, etc.). When a sporulating bacterium is employed, the desired protein is included in the resultant spores when such bacteria are induced to sporulate. When gram-negative bacteria are employed, the desired protein is secreted into the periplasm, from which it can be isolated or substantially purified by known methods.

When a bacterium is employed in the inventive method, it must produce a functional SipW protein. While some species of Bacillus (e.g., B. subtilis) produce a native SipW protein, when other bacteria are used, they are induced to produce SipW by introducing into them a gene that encodes a functional SipW protein (such as described above). Prokaryotic promoters for expressing genes in sporulating bacteria are known in the art, and a preferred promoter is a regulatable promoter (e.g., pSPAC, see Yansura et al., Proc. Natl. Acad. Sci. (U.S.A.), 81, 439-43 (1984)). Within the cassette, such promoters are operably linked to the SipW gene.

When a sporulating bacterium is employed in the inventive method, typically it will be a species of either Clostridium or Bacillus, which produces spores under varying environmental conditions. Generally, the species and strain are selected depending on the desired properties of the spore. For example, it is known that Clostridium species generally are more tolerant to anaerobic conditions than Bacillus, and various strains and species are more tolerant to heat than others. Thus, it is within the routine skill of the art to select a desired species and strain of sporulating bacterium for use in the inventive method. Many standard laboratory strains suitable for use in the inventive method are known in the art (e.g., 168, PY79, etc.). The conditions for sporulating bacteria such as Clostridium and Bacillus are known generally to involve depriving the host cells of nutrients. Any suitable protocol can be employed in the inventive method. During sporulation in the presence of SipW, the SipW recognition domain is cleaved from the immature fusion protein, and the mature protein is exported from the cell. Because the spore septum forms during this period, the mature (i.e., processed) protein of interest is incorporated into the spore as well as secreted into the supernatant culture medium.

The inventive method thus results in spores containing the desired protein. Such spores have many end uses, such as in medicine, agriculture, waste treatment, etc. For example, when introduced into an animal, the spores slowly degrade. Because of their resiliency, the protein of interest will remain in contact with the tissues of the animal for a longer period of time (e.g., have a longer residence time) than if the isolated protein were introduced into the animal. Thus, spores prepared in accordance with the inventive method can be employed as a type of time-release device for exposing the animal to the protein of interest over a prolonged period. This will enable physiologically-active proteins (e.g., enzymes) to operate longer within the animal. Moreover, such spores enable antigenic proteins (e.g., elements of vaccines) to be delivered to the animal over a prolonged period for more efficient inoculation. Additionally, as bacterial spores display selective tropism for certain tumors, spores prepared in accordance with the inventive method can deliver antitumor agents (e.g., toxins, enzymes, anti-angiogenic factors, etc.) to tumors as part of a therapeutic regimen for combating the tumors. For use in agriculture, the spores can be engineered to contain insecticides to control insect populations. Other spores can be engineered to contain enzymes for degrading natural or industrial waste or to aid in soil or waste treatment protocols using sporulating bacteria, or, for example in activated sludge.

To facilitate the use of spores or of secreted TasA protein, they can be formulated into suitable compositions, e.g., pharmacological or agricultural compositions. Generally, such compositions include the active ingredient (i.e., the spore or the TasA protein or the active fragment thereof) and a dispersant or carrier, e.g., a pharmacologically or agriculturally acceptable carrier. Such compositions can be suitable for delivery of the active ingredient to a patient for medical application, for delivery of the active ingredient for agricultural or ecological control, etc. Such compositions can be manufactured in a manner that is itself known, e.g., by means of conventional mixing, dissolving, granulating, dragee-making, levigating, emulsifying, encapsulating, entrapping or lyophilizing processes.

Pharmacological (e.g., pharmaceutical) compositions for use in accordance with the present invention can be formulated in a conventional manner using one or more pharmacologically or physiologically acceptable carriers comprising excipients, as well as optional auxiliaries which facilitate processing of the active compounds into preparations which can be used pharmaceutically. Proper formulation is dependent upon the route of administration chosen. Thus, for injection, the active ingredient can be formulated in aqueous solutions, preferably in physiologically compatible buffers. For transmucosal administration, penetrants appropriate to the barrier to be permeated are used in the formulation. Such penetrants are generally known in the art. For oral administration, the active ingredient can be combined with carriers suitable for inclusion into tablets, pills, dragees, capsules, liquids, gels, syrups, slurries, suspensions, and the like. For administration by inhalation, the active ingredient is conveniently delivered in the form of an aerosol spray presentation from pressurized packs or a nebuliser, with the use of a suitable propellant. The active ingredient can be formulated for parenteral administration by injection, e.g., by bolus injection or continuous infusion. Such compositions can take such forms as suspensions, solutions or emulsions in oily or aqueous vehicles, and can contain formulatory agents such as suspending, stabilizing, and/or dispersing agents. Other pharmacological excipients are known in the art.

Other compositions can be formulated for delivering the antibiotic or spores to plants, fields, lawns, orchards, lakes or other agricultural targets. Examples of suitable agricultural compositions are known in the art and include, for example, wettable powders, dry flowables, microencapsulation of effective agents, liquid or solid formulations and antibiotic fractions obtained from suitable cultures (see, e.g., U.S. Pat. Nos. 5,061,495 and 5,049,379). Such compositions can be formulated in powder, granular, pellet or bait form with solid carriers. Alternatively, such compositions can be formulated as liquids for dusting or spraying.

While it is believed that one of skill in the art is fully able to practice the invention after reading the foregoing detailed description, the following examples further illustrate some of its features. In particular, they demonstrate the antibiotic properties of the TasA protein and SipW-dependent secretion of proteins from disparate bacterial systems. As these examples are included for purely illustrative purposes, they should not be construed to limit the scope of the invention in any respect.

The procedures employed in these examples, such as affinity chromatography, Southern blots, PCR, DNA sequencing, vector construction (including DNA extraction, isolation, restriction digestion, ligation, etc.), bacterial culture (including antibiotic selection and sporulation), transfection or transformation of bacteria, protein assays (e.g., SDS-PAGE, Western blotting), etc. are techniques routinely performed by those of skill in the art (see gene-rally Sambrook et al., Molecular Cloning, A Laboratory Manual, Cold Spring Harbor Laboratory, Cold Spring Harbor, N.Y. (1989)). Accordingly, in the interest of brevity, the experimental protocols are not discussed in detail.

### EXAMPLE 1

This example demonstrates the identification of the TasA and YqxM genes and proteins and identify then as secreted proteins.

Spores were prepared from wild-type B. subtilis as well as from strains bearing a mutation either in gerE (spores lacking the inner coat and much of the outer coat), cotE (spores lacking the outer coat) or in both genes (spores lacking both coat layers). Proteins were extracted from these spores and examined by SDS-PAGE.

While several proteins were present, the analysis revealed high amounts of a 31 kDa species in spores lacking a fully assembled coat (from gerE and gerE/cotE mutant strains). Small amounts of the 31 kDa protein were detected in extracts from wild type spores and spores lacking an outer coat (from a cotE strain), while a greater amount of it was detected in extracts of spores deficient in the inner coat (from a gerE or a gerE/cotE mutant strain). Additionally, a band with identical mobility on SDS-PAGE was detected within a lysate of sporangia at hour five of sporulation from which spores and cell debris were removed by centrifugation, suggesting a non-spore-associated form of this protein. The higher extractability of the 31 kDa protein from spores lacking a fully assembled coat is consistent with an interior location for the 31 kDa species, as the coat layers act as barriers that inhibit the extraction of proteins.

To identify the gene encoding the 31 kDa species, the protein was isolated from the gel and subjected to limited N-terminal amino acid sequencing by Edman degradation. Nine N-terminal amino acids from the non-spore-associated 31 kDa band were identified, and a search of the B. subtilis genome (Kunst, et al., Nature, 390, 249-56 (1997)) revealed a perfect match of this amino acid sequence to only one open reading frame: yqhF, encoding a hypothetical protein of 28153.9 Da (261 amino acid residues), referred to herein as TasA.

To confirm that this gene encoded the 31 kDa species, a TasA deletion strain was constructed, and a rabbit polyclonal anti-serum was generated against the non-spore-associated 31 kDa species. This anti-serum reacted to both the 31 kDa protein extracted from intact spores and the non-spore-associated protein from a lysate that had been cleared of spores, as assayed by Western analysis. As with SDS PAGE analysis, this western analysis detected more spore-associated protein in spores missing most or all of the coat (due to gerE or gerE/cotE mutations) than wild type cells. Pre-immune serum was used as a control, which did not recognize the 31 kDa protein. Moreover, no staining was observed when post-immune serum was used to probe spore extracts prepared from the TasA deletion strain. These data suggest that the spore-associated and the non-spore-associated 31 kDa proteins are the product of the same gene. Similar experiments identified YqxM (SEQ ID NO:4) as a 34 kDa SipW-dependent spore-associated protein that is cleaved at amino acid 44.

That the N-terminus portion of the protein is cleaved suggests that the protein is secreted from cells during sporulation. To test this view, culture supernatants were prepared two hours after the onset of sporulation, concentrated by ethanol precipitation, and subjected to Western blot analysis to probe for TasA. This analysis revealed that TasA was present in the supernatants of sporangia at this time point while no signal was detected in supernatants of sporangia lacking the TasA gene. These results indicate that TasA is exported from cells during sporulation.

### EXAMPLE 2

This example demonstrates that the TasA protein is an antibiotic.

To test the antibiotic activity of TasA, recombinant TasA was produced in transformed E. coli and applied to a battery of bacteria (indicated in Table 1) on either LB, King's B or Mueller-Hinton plates. A swab was placed in a culture at 0.5 MacFarland units and used to inoculate agar plates with the bacteria. After plating the cells, a cork borer (12 mm diameter) was employed to punch two holes into the agar-plates and remove an agar disc. Subsequently, 400 μl of crude lysate from the TasA-expressing E. coli strain or a non-expressing control strain were added to the holes of each plate. After 16 to 24 hours the diameter of the clear zone around each hole was measured.

In the case of strains resistant to TasA, no clear zone could be detected surrounding the wells. In the case of strains susceptible to TasA, a clear zone surrounding the wells could be detected. Some strains appeared to be susceptible to the lysate of the control strain lacking any TasA, and the control well was surrounded by a clear zone. Strains that were resistant to TasA displayed a zone surrounding both wells, and strains that were sensitive displayed a larger zone surrounding the well with the lysate of the TasA overproducing strain. These results, summarized in Table 1, demonstrate that the TasA protein has a specific antibiotic profile.

TABLE 1

| Bacterial Strain | Response |
|---|---|
| Agrobacterium tumifaciens GV3101 | susceptible |
| Erwinia amylovora EG321 | susceptible |
| Erwinia chrysanthamum ACH150 | susceptible |
| Pseudomanas syringae pv. tomato DC3000 | susceptible |
| Klebsiella pneumoniae A95 | susceptible |
| Pseudomonas aurofaciens | susceptible |
| Pseudomanas syringae pv. syringae 61 | susceptible |
| Erwinia A1750 | susceptible |
| Pseudomonas putida | susceptible |
| Agrobacterium tumafaciens GV3101 | resistant |
| Pseudomanas aeruginosa PAO | resistant |
| E. coli 35218 | susceptible |
| E. coli 25922 | susceptible |
| Streptococcus bovis 9809 | resistant |
| Streptococcus pyogenes 19615 | resistant |
| Pseudomonas aeruginosa 27853 | resistant |
| Klebsiella pneumoniae 13883 | susceptible |
| E. coli 773465 | susceptible |
| E. coli 773813 | susceptible |
| E. coli 773671 | susceptible |
| Staphylococcus X54017* | susceptible |
| Staphylococcus 174812* | susceptible |
| Staphylococcus X54017* | susceptible |
| Klebsiella pneumoniae 573266 | resistant |
| Klebsiella pneumoniae 773813 | susceptible |
| Klebsiella pneumoniae 773465 | susceptible |
| Enterobacter cloacae 538252-2 | susceptible |
| Enterobacter cloacae 618690 | susceptible |
| Staphylococcus aureus S73185^{#} | resistant |
| Xanthomonas campestris (BP109) | susceptible |

*= Coagulase negative, species not identified.
#= Methicillin-resistant

### EXAMPLE 3

This example demonstrates the activity of SipW as a signal peptidase responsible for secreting proteins into a forming Bacillus spore.

To test the involvement of sip Win TasA maturation, strains in which sip W was deleted but TasA would still be expressed were generated. A portion of the sipW open reading frame was replaced with a neomycin resistance cassette (Itaya et al., NAR, 17, 4410 (1989)); strains were constructed in which the neomycin resistance cassette was oriented in both possible directions. Western blot analysis was then used to examine the lysates of vegetative cells, sporulating cells, and spores of cells bearing these mutations. Based on the deduced amino acid sequence, the immature form of TasA was expected to exhibit a molecular weight of approximately 34 kDa, i.e., 3 kDa larger than the mature form.

In cells bearing the neomycin resistance cassette oriented against the predicted direction of TasA transcription, no TasA signal was detected either in sporangial lysates or in whole vegetative cell lysates. In contrast, a 34 kDa band was detected in both sporangial lysates and vegetative cells, from cells bearing the neomycin resistance cassette oriented towards the predicted direction of TasA transcription consistent with the constitutive activity of the neo promoter.

That this band was larger than the 31 kDa mature form suggests that SipW is required for proper processing of TasA. Moreover, the absence of the 34 kDa band from the supernatant fraction is consistent with the role of SipW in secretion.

Similar experiments were conducted to determine the requirement of SipW for secretion of the YqxM protein. As was the case with TasA, deletion of sipW resulted in no measurable amount of YqxM associated with the spore. Placing SipW under the control of the pSpac promoter resulted sipw-dependent YqxM secretion.

To determine if factors other than SipW are required to process TasA, the neomycin resistance gene was inserted into YqxM. This mutation destroyed the coding sequence of YqxM but allowed the neomycin resistance gene promoter to direct expression of both sipW and TasA. Western blot analysis was then employed to determine if cells of this strain produced mature TasA (31 kDa) during vegetative growth and during sporulation. A band corresponding to mature TasA was detected in extracts of spores and in both the culture supernatants and lysates of sporangia and vegetative cells. No protein of larger molecular weight that could correspond to immature TasA was detected.

These data indicate that YqxM and TasA can be translocated across membranes in a sipW-dependent manner. Moreover, sipW appears to be required for the incorporation of TasA into the spore. Moreover, SipW appears to be the only sporulation-specific cellular factor required for cleavage and secretion of TasA and YqxM.

### EXAMPLE 4

This example demonstrates the SipW-dependent secretion of YqxM from E. coli.

E. coli strains were constructed that over-produced either YqxM and SipW together, or YqxM alone. Cell extracts and concentrated culture supernatants were assessed by Western hybridization to determine the presence and mobility of YqxM in these strains.

Bands of 38 kD and approximately 33kD were detected in cell extracts from the strain producing YqxM alone, the smaller band likely representing proteolytic cleavage. These bands were present even in the absence of IPTG (although at lower intensity), indicating sufficient promoter activity to permit detectable YqxM synthesis even in the absence of induction. A single band of 38 kD was detected in cell extracts from the strain expressing yqxM and sipW, indicating that sipW was responsible for a decrease in the molecular mass of YqxM, a result consistent with the cleavage of the putative signal peptide.

Examination of concentrated E. coli culture supernatants by western blot analysis revealed that although YqxM was produced without IPTG, YqxM was released into the medium only when IPTG was present. In the strain producing YqxM alone, the level of YqxM in the culture supernatant was relatively low and appeared similar in mobility to the presumed YqxM degradation product. However, when both YqxM and SipW were produced, YqxM was abundant in the culture supernatant and migrated as a smaller 31 kD species.

These data demonstrate that SipW is able to function properly in membrane systems other than Bacillus.

### EXAMPLE 5

This example demonstrates the SipW-dependent secretion of a chimeric protein into a Bacillus spore.

A SipW-dependent signal peptide is fused to the open reading of a gene encoding a cytoplasmic protein not normally secreted from the cell. Specifically, using PCR, a region of the B. subtilis chromosome including the sipW gene, the gene segment of TasA that encodes its signal peptide, and the promoter that controls the expression of the TasA gene is amplified. This fragment is then fused to a PCR product that encodes the entire open reading frame of an epitope tagged version of the cotE gene (Zheng, et al., Genes & Dev., 2, 1047-54 (1988)). Translation of this construct results in the production of the SipW protein and a tagged CotE protein having the TasA signal peptide.

The construct is then cloned into the B. subtilis chromosome to replace the native sipW/TasA cassette. Secreted proteins are thereafter monitored by Western analysis for the presence of the epitope tagged version of CotE. Proteins in the supernatants of cells lacking this construct serve as a control. The epitope-tagged CotE protein is detectable only in the supernatant of cells that have been transformed with the construct and in the spores produced from these cells.

All of the references cited herein, including patents, patent applications, and publications, are hereby incorporated in their entireties by reference.

While this invention has been described with an emphasis upon preferred embodiments, it will be obvious to those of ordinary skill in the art that variations of the preferred embodiments may be used and that it is intended that the invention may be practiced otherwise than as specifically described herein. Accordingly, this invention includes all modifications encompassed within the spirit and scope of the invention as defined by the following claims.

What is claimed is:

1. An isolated or substantially purified Bacillus TasA protein having the following physicochemical properties:
(a) retards the growth of at least 80% of the following microbes: Microccocus luteus (4698), Staphylococcus epidermis (12228), Enterococcus faecalis (29212), E. coli (35218), E. coli (25922), Klebsiella pneumoniae (13883), E. coli (773465), E. coli (773813), E. coli (773671), Coagulase negative Staphylococcus sp. (X54017), Coagulase negative Staphylococcus sp. (174812), Coagulase negative Staphylococcus sp. (X54017), Klebsiella pneumoniae (773813), Klebsiella pneumoniae (773465), Enterobacter cloacae (538252-2), Enterobacter cloacae (618690), Agrobacterium tumifaciens (GV3101), Erwinia amylovora (EG321), Erwinia chrysanthamum (ACHI 50), Pseudomanas syringae pv. tomato (DC3000), Klebsiella pneumoniae (A95), Pseudomonas aurofaciens, Pseudomanas syringae pv. syringae (61), Erwinia (A1750), Pseudomonas putida, and Xanthomonas campestris (BP109), and
(b) molecular weight of about 31 kDa as measured by denaturing SDS polyacrylamide gel electrophoresis.

2. The protein of claim 1, wherein the protein consists essentially of the sequence of SEQ ID NO:2 and/or a conservative variation thereof.

3. The protein of claim 1, which is recombinant.

4. The protein of claim 1 comprising SEQ ID NO:2.

5. An organism harboring a recombinant vector comprising a nucleic acid encoding the protein of claim 1.

6. The organism of claim 5, which expresses the nucleic acid to produce an antibiotic protein in biologically active form.

7. A composition comprising a pharmacologically or agriculturally acceptable carrier and the protein of claim 1, or active fragment thereof.

9. A method of killing an insect, the method comprising exposing the insect to the protein of claim 1 so as to kill the insect.

10. A method of treating a microbial culture, the method comprising exposing the culture to the protein of claim 1 so as to inhibit the growth of the culture.

11. An isolated or substantially purified protein which contains a sequence of amino acids at least 70% identical to SEQ ID NO:2 and which retards the growth of one or more microbes selected from the group of microbes consisting of: Microccocus luteus (4698), Staphylococcus epidermis (12228), Enterococcus faecalis (29212), E. coli (35218), E. coli (25922), Klebsiella pneumoniae (13883), E. coli (773465), E. coli (773813), E. coli (773671), Coagulase negative Staphylococcus sp. (X54017), Coagulase negative Staphylococcus sp. (174812), Coagulase negative Staphylococcus sp. (X54017), Klebsiella pneumoniae (773813), Klebsiella pneumoniae (773465), Enterobacter cloacae (538252-2), Enterobacter cloacae (618690), Agrobacterium tumifaciens (GV3101), Erwinia amylovora (EG321), Erwinia chrysanthamum (ACH150), Pseudomanas syringae pv. tomato (DC3000), Klebsiella pneumoniae (A95), Pseudomonas aurofaciens, Pseudomanas syringae pv. syringae (61), Erwinia (A1750), Pseudomonas putida, and Xanthomonas campestris (BP109).

12. The protein of claim 11, which is recombinant.

13. The protein of claim 11 comprising SEQ ID NO:2.

14. An organism harboring a recombinant vector comprising a nucleic acid encoding the protein of claim 11.

15. The organism of claim 14, which expresses the nucleic acid to produce an antibiotic protein in biologically active form.

16. A composition comprising a pharmacologically or agriculturally acceptable carrier and the protein of claim 11, or active fragment thereof.

17. A method of killing an insect, the method comprising exposing the insect to the protein of claim 11 so as to kill the insect.

18. A method of treating a microbial culture, the method comprising exposing the culture to the protein of claim 1 so as to inhibit the growth of the culture.

19. A method of producing a desired protein, the method comprising (a) introducing an expression cassette into a membrane-bound genetic system comprising a membrane separating the system into at least two compartments, a first of the compartments containing transcription machinery, translation machinery, and a SipW protein, the expression cassette including a nucleic acid encoding a fusion protein comprising a SipW recognition sequence domain and a domain consisting of the desired protein, (b) directing genetic transcription and translation within the first of the compartments to produce the fusion protein within the first of the compartments, and (c) causing the SipW protein to recognize the SipW recognition sequence domain, whereby the desired protein is translocated across the membrane into a second of the compartments.

20. The method of claim 19, wherein the genetic system comprises a sporulating bacterium, and wherein the bacterium is induced to sporulate, whereby the desired protein is incorporated into the resulting spores.

21. A spore prepared in accordance with the method of claim 20.

22. A composition comprising the spore of claim 21 and a pharmacologically or agriculturally acceptable carrier.

23. An isolated nucleic acid encoding a fusion protein comprising a first domain and a second domain comprising at least 15 residues from the amino terminus of SEQ ID NO:2 or SEQ ID NO:4.

24. A recombinant expression cassette comprising the isolated nucleic acid of claim 24 operably linked to a prokaryotic promoter.

25. A bacterium harboring the recombinant expression cassette of claim 24.

26. The bacterium of claim 25, which is a species of Clostridium or Bacillus.

27. The bacterium of claim 25, which produces a SipW protein.

28. An isolated nucleic acid encoding a SipW protein.

29. The isolated nucleic acid of claim 24 operably linked to a prokaryotic promoter.

30. A bacterium harboring the recombinant expression cassette of claim 28.

31. The bacterium of claim 29, which is a species of Clostridium or Bacillus.

32. The bacterium of claim 29, which produces a SipW protein.
